(19) United States
(12) Patent Application Publication
(10) Pub. No.: US 2014/0243698 A1
(43) Pub. Date: Aug. 28, 2014

(54) SENSOR ADAPTOR, APPARATUS, AND METHOD FOR MONITORING END-TIDAL CARBON DIOXIDE

(71) Applicant: Carol Koch, Riverview, MI (US)

(72) Inventor: Carol Koch, Riverview, MI (US)

(21) Appl. No.: 14/159,932

(22) Filed: Jan. 21, 2014

Related U.S. Application Data
(60) Provisional application No. 61/768,620, filed on Feb. 25, 2013.

(51) Int. Cl. A61M 16/08; A61M 16/10; A61M 16/06; A61B 5/083; A61B 5/097

(52) U.S. Cl. CPC A61M 16/085; A61B 5/0836; A61B 5/097; A61M 16/06; A61M 16/0875; A61M 16/1005
USPC 600/531; 29/428

(57) ABSTRACT

A sensor adaptor (20) couples a face mask (22) to a gas sampling tube (24) connected to a device detecting end-tidal carbon dioxide. The sensor adaptor (20) includes a shaft (30) and connector (54). The shaft (30) includes a channel (40) providing a pathway for the carbon dioxide to travel toward the gas sampling tube (24). One end of the shaft (30) includes an adaptor tip (26) which extends through an exit port (28) of the face mask (22). A connector (54) is attached to the other end of the shaft (30) and couples the sensor adaptor (20) to the gas sampling line. A gripper (42) may surround the shaft (30) and prevent improper advancement of the shaft (30) into the face mask (22). The sensor adaptor (20) can be designed for use with any gas sampling tube and any face mask including exit ports.

### CROSS REFERENCE TO RELATED APPLICATION

This U.S. utility patent application claims the benefit of U.S. provisional patent application No. 61/768,620, filed Feb. 25, 2013, the content of which is incorporated herein by reference in its entirety.

### BACKGROUND OF THE INVENTION

1. Field of the Invention

The invention relates generally to an apparatus for improving detection of the presence of a patient's end-tidal carbon dioxide, a method of forming the apparatus, and a method for improving detection of end-tidal carbon dioxide.

2. Related Art

In the field of health care, patients under the care of a practitioner oftentimes require oxygen delivery. Various different techniques can be used to deliver oxygen to the patient. One technique includes the use of a face mask, such as a simple face mask, with an opening in the nose region for receiving an oxygen delivery tube. When the face mask is used to deliver oxygen, the patient's exhaled carbon dioxide, referred to as end-tidal carbon dioxide (CO₂), must be monitored. The presence or absence of end-tidal carbon dioxide provides valuable information to the practitioner. For example, the absence of end-tidal carbon dioxide indicates there could be an obstruction in the patient's airway, which could threaten the patient's health or safety. Thus, it a reliable method of monitoring end-tidal carbon dioxide is necessary.

One technique currently used to detect end-tidal carbon dioxide includes the use of an anesthesia machine, for example a D-fend™. The practitioner manually lifts the face mask slightly off the patient's face, and places a gas sampling tube connected to the machine underneath the face mask. However, when this technique is used, the practitioner must manually hold the gas sampling tube in place, or carefully watch the patient to make sure the gas sampling tube remains in the correct position. The gas sampling tube must not obstruct oxygen delivery or slip out from under the face mask. If the gas sampling tube is moved from the correct position, the anesthesia machine is unable to accurately detect the presence or absence of the patient's end-tidal carbon dioxide, and the patient's health and safety could be at risk. There is clearly a need for more convenient and reliable methods of detecting a patient's end-tidal carbon dioxide.

### SUMMARY OF THE INVENTION

One aspect of the invention comprises a sensor adaptor which provides a secure, convenient, and reliable connection between a face mask and a gas sampling tube connected to a device detecting the presence or absence of the patient's end-tidal carbon dioxide. The sensor adaptor includes a shaft and a connector. The shaft extends from a first end to a second end and includes a shaft outer surface presenting a shaft outside diameter. The shaft outside diameter is not greater than an exit port diameter of the face mask for which the sensor adaptor is designed. The shaft also includes a shaft inner surface presenting a channel extending continuously from the first end to the second end for providing a continuous pathway from the underside of the face mask toward the gas sampling tube. The shaft also includes an adaptor tip adjacent the first end, which is disposed in the exit port of the face mask. The connector is attached to the shaft for coupling the channel of the shaft to the gas sampling tube.

In one embodiment, the sensor adaptor includes a gripper surrounding the shaft adjacent the first end and presenting the adaptor tip between the gripper and the first end of the shaft. The gripper includes a gripper outer surface presenting a gripper outside diameter which is greater than the shaft outside diameter.

Another aspect of the invention provides an apparatus for coupling to a device detecting the presence or absence of the patient's end-tidal carbon dioxide. The apparatus includes the face mask for disposing over the nose and mouth of a patient. The face mask includes a mask inner surface presenting an inner volume. The face mask also includes a nasal opening for receiving an oxygen delivery tube and a plurality of exit ports for allowing carbon dioxide to exit the inner volume. Each of the exit ports presents an exit port diameter. The apparatus also includes the sensor adaptor for coupling the face mask to a gas sampling tube connected to the device detecting the end-tidal carbon dioxide. The adaptor tip of the sensor adaptor is disposed in one of the exit ports of the face mask, and the shaft outside diameter is not greater than the exit port diameter of the one exit port in which the shaft is disposed.

Yet another aspect of the invention provides a method for manufacturing the apparatus for coupling to the device detecting the presence or absence of end-tidal carbon dioxide. The method includes providing the face mask, and disposing the sensor adaptor in one of the exit ports of the face mask. According to one embodiment, the step of disposing the sensor adaptor in one of the exit ports of the face mask includes inserting the adaptor tip into the exit port until the gripper engages the mask outer surface.

Another aspect of the invention provides a method for improving detection of the presence or absence of the patient's end-tidal carbon dioxide. The method includes providing the face mask and the sensor adaptor, and coupling the sensor adaptor to the face mask by inserting the adaptor tip through one of the exit ports of the face mask until the stopper end engages a mask outer surface. The method also includes coupling the oxygen delivery tube to the nasal opening of the face mask, and disposing the face mask over the nose and mouth of the patient. The method further includes coupling the connector end of the sensor adaptor to the gas sampling tube, which is connected to the device detecting the end-tidal carbon dioxide.

The sensor adaptor of the present invention provides numerous advantages. For example, the sensor adaptor can be used with any existing face mask including exit ports, such as the simple face mask. The adaptor tip and the exit port provide a tight frictional engagement or press fit therebetween. Thus, the sensor adaptor remains in the exit port of the face mask, even while patient moves about or moves the face mask, and provides a secure connection between the inner volume of the face mask, where the patient exhales carbon diode, and the gas sampling tube. In addition, the sensor adaptor does not obstruct oxygen delivery or prevent carbon dioxide from exiting the mask. If the practitioner needs to remove the sensor adaptor, he or she simply pulls the adaptor tip of the sensor adaptor out of the exit port. The reliability provided by the sensor adaptor allows for more rapid detection of an obstructed airway, or other potential safety issues, and thus improves the quality of care provided to the patient.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a perspective view of a sensor adaptor according to one exemplary embodiment of the invention;

FIG. 2 is an exploded view of the sensor adaptor of FIG. 1 adjacent an end of an exemplary gas sampling tube;

FIG. 3 is a perspective view of a sensor adaptor according to another exemplary embodiment of the invention; and

FIG. 4 is a perspective view of the sensor adaptor of FIGS. 1 and 2 connected to the gas sampling tube and inserted in an exit port of a face mask.

### DESCRIPTION OF THE ENABLING EMBODIMENT

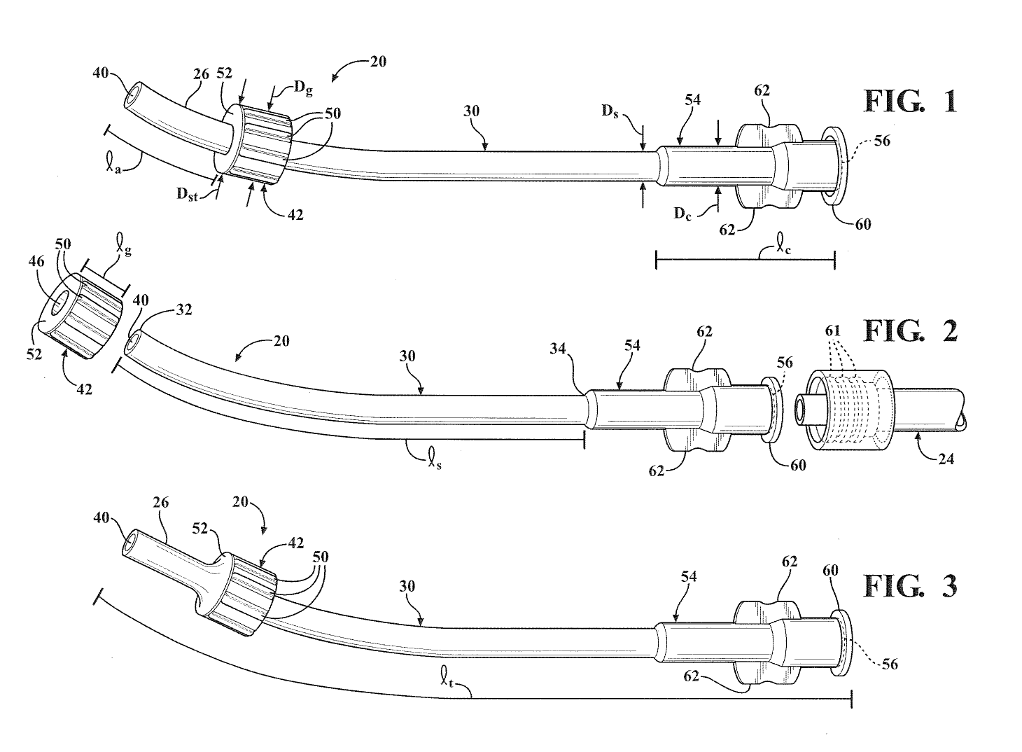

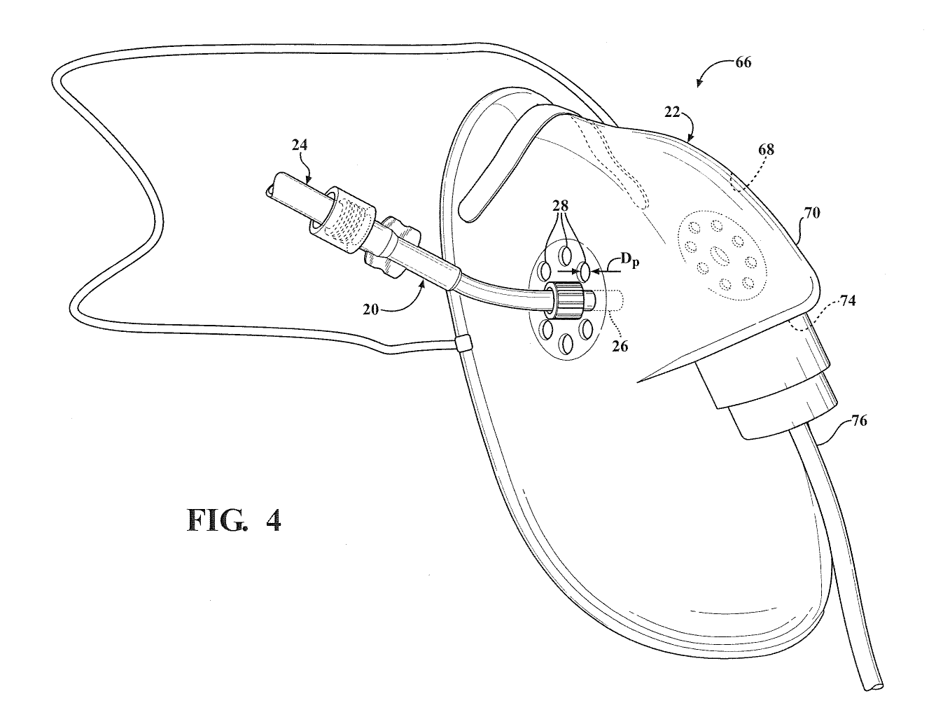

A sensor adaptor 20 for coupling a face mask 22 to a gas sampling tube 24, which is connected to a device detecting the presence and/or absence of a patient's end-tidal carbon dioxide, is generally shown in FIGS. 1-3. An adaptor tip 26 of the sensor adaptor 20 can be inserted through an exit port 28 of the face mask 22 to maintain a secure connection between the underside of the face mask 22 and the gas sampling tube 24, as shown in FIG. 4. The sensor adaptor 20 provides a quicker, more efficient, and more reliable method of detecting end-tidal carbon dioxide, compared to prior art methods of monitoring end-tidal CO₂, such as manually inserting an end of the standard gas sampling tube 24 underneath the face mask 22 and monitoring the patient to make sure the standard gas sampling tube 24 stays in position while the patient breathes.

The sensor adaptor 20 includes a shaft 30 extending from a first end 32 to a second end 34 and a shaft length Iₛ extending from the first end 32 to the second end 34. The shaft 30 includes a shaft outer surface which typically presents a cylindrical shape and has a shaft outside diameter Dₛ. The shaft outside diameter Dₛ is designed so that it is not greater than an exit port diameter Dₚ of one of the exit ports 28 of the face mask 22, through which the sensor adaptor 20 is inserted. This can be any one of the many exit ports 28 present in a face mask 22. Typically, the shaft outside diameter Dₛ is just slightly less than the exit port diameter Dₚ, so that the practitioner needs to exert a small amount of force to insert or remove the shaft 30 from the exit port 28. In one exemplary embodiment, the shaft outside diameter Dₛ is approximately 0.1 to 0.5 cm, for example 0.3 cm. The shaft 30 also includes a shaft inner surface facing opposite the shaft outer surface. The shaft inner surface presents a channel 40 also typically having a cylindrical shape. The channel 40 is unobstructed and extends continuously from the first end 32 to the second end 34 of the shaft 30 to provide a direct pathway toward the gas sampling tube 24. The size and geometry of the channel 40 can vary for any reason, for example to provide the most efficient detection method.

The shaft 30 also includes an adaptor tip 26 adjacent the first end 32, which is inserted through the exit port 28 of the face mask 22. The adaptor tip 26 also presents the channel 40 providing the pathway toward the gas sampling tube 24. The adaptor tip 26 presents an adaptor length Iₐ being a portion of the shaft length Iₛ. In the exemplary embodiment, the adaptor length Iₐ is 0.3 cm to 1.0 cm, for example 0.5 cm. However, the adaptor length Iₐ can vary. For example, the adaptor length Iₐ can vary depending on the geometry and thickness of the face mask 22, or for other reasons.

The sensor adaptor 20 also typically includes a gripper 42 surrounding the shaft 30 adjacent the first end 32. However, the sensor adaptor 20 can be formed without the gripper 42. When present, the gripper 42 can comprise any shape. In the exemplary embodiment, the gripper 42 includes a gripper inner surface presenting a gripper opening 46 surrounding the shaft 30. The gripper 42 also includes a gripper outer surface presenting a gripper outside diameter D_{g} which is greater than the shaft outside diameter Dₛ. The gripper 42 presents a gripper length I_{g} extending along a portion of the shaft length Iₛ. In the exemplary embodiment, the gripper length I_{g} is 0.5 cm to 1.5 cm, for example 1.0 cm. The gripper outer surface presents a plurality of grooves 50 extending parallel to the shaft 30 for allowing a practitioner to grip the sensor adaptor 20. The design of the grooves 50 can vary as desired. The gripper 42 allows the practitioner to easily control and position the sensor adaptor 20 relative to the face mask 22. In the embodiment of FIGS. 1, 2 and 4, the gripper 42 is manufactured separate from the shaft 30, and the first end 32 of the shaft 30 is inserted through the gripper opening 46 to form the sensor adaptor 20. In this embodiment, the gripper 42 and the shaft 30 present a tight frictional engagement or press fit therebetween. In the embodiment of FIG. 3, the gripper 42 and the shaft 30 are formed integral with one another, for example by a single molding process.

The gripper 42 of the sensor adaptor 20 also includes a stopper end 52. The stopper end 52 comprises a surface facing generally toward the first end 32 of the shaft 30 for preventing improper advancement of the sensor adaptor 20 into the face mask 22. The stopper end 52 and the first end 32 of the shaft 30 present the adaptor tip 26 therebetween. The surface of the stopper end 52 can be planar and perpendicular to the shaft 30, as shown in FIGS. 1, 2, and 4, or disposed at another angle relative to the shaft 30, or slightly curved, as shown in FIG. 3. The surface of the stopper end 52 has a stopper diameter Dₛₜ which is greater than the shaft outside diameter Dₛ. In the embodiments of FIGS. 1-4, the gripper 42 and the stopper end 52 are formed as a single component. However, the gripper 42 and the stopper end 52 could comprise separate components.

The sensor adaptor 20 also includes a connector 54 attached to the second end 34 of the shaft 30 for coupling the shaft 30 to the gas sampling tube 24. The connector 54 is typically separate from the gas sampling tube 24, but it could be formed integral with the gas sampling tube 24. Various different types of connectors 54 can be used. In the exemplary embodiment, the connector 54 includes a female Luer lock and the gas sampling tube 24 includes a male Luer, as shown in FIGS. 2 and 4. In this embodiment, the connector 54 of the adaptor presents a connector opening 56 which is in fluid communication with the channel 40 of the shaft 30. The connector 54 also has a connector end opposite the shaft 30 which is open so that an end of the gas sampling tube 24 can be received in the connector opening 56. At least one thread 60 extends radially outwardly from the connector 54 for engaging threads 61 of the male Luer lock at the end of the gas sampling tube 24. The connector 54 of the exemplary embodiment also includes a pair of wings 62 extending longitudinally along and radially outwardly, which can be gripped by the practitioner while attaching the female Luer lock to the male Luer lock. However, the connector 54 can alternatively be formed without the wings 62. The connector 54 also includes a connector outer surface presenting a connector outside diameter D_{c} which is greater than the shaft outside diameter Dₛ. In the exemplary embodiment, the connector 54 presents a connector length I_{c} being 2.0 cm to 2.8 cm, for example 2.4 cm, and the distance between the connector 54 and the gripper 42 is 1.5 cm to 2.5 cm, for example 2.0 cm. However, the connector length I_{c} can vary for any reason.

The sensor adaptor 20 presents a total length Iₜ extending from the open connector end to the first end 32 of the shaft 30. In the exemplary embodiment, the total length Iₜ of the sensor adaptor 20 is 5.0 cm to 6.5 cm, for example 5.9 cm. However, the total length Iₜ of the sensor adaptor 20 can vary. The components of the sensor adaptor 20 are preferably each formed of a translucent or transparent plastic material, for example clear material, but they could be formed of other materials having various different colors. As discussed above, the components of the sensor adaptor 20 are preferably formed integral with one another, such that the sensor adaptor 20 comprises a single piece. The components of the sensor adaptor 20 could alternatively be formed separate from one another, and then connected together.

Another aspect of the invention provides an apparatus 66 for coupling to the device which detects at least one of the presence and absence of end-tidal carbon dioxide. The apparatus 66 includes the face mask 22 which is disposed over the nose and mouth of the patient. Various different types of face masks 22 can be used. However, in the exemplary embodiment shown in FIG. 4, the face mask 22 is a simple face mask. The face mask 22 can be formed of various different materials, but is preferably formed of a translucent or transparent plastic material. The face mask 22 includes a mask inner surface 68 which faces the nose and mouth of the patient and a mask outer surface 70 facing opposite the mask inner surface 68. As shown in FIG. 4, the mask inner surface 68 has a contour resembling the contour of the nose and mouth of the patient. The mask inner surface 68 also presents an inner volume between the mask inner surface 68 and the nose and mouth of the patient. When the patient exhales carbon dioxide, the carbon dioxide enters this inner volume.

The face mask 22 also includes a nose portion with a nasal opening 74 for receiving an oxygen delivery tube 76, as shown in FIG. 4, which delivers oxygen to the patient. The oxygen delivery tube 76 presents an airway being open for allowing oxygen to enter the inner volume of the face mask 22 and become accessible to the nose and mouth of the patient. The face mask 22 also includes a pair of side portions disposed on opposite sides of the nose portion. Each of the side portions includes a plurality of the exit ports 28 for allowing the end-tidal carbon dioxide to exit the inner volume. As discussed above, each of the exit ports 28 presents an exit port diameter Dₚ, and at least one of the exit ports 28 has an exit port diameter Dₚ which is slightly greater than or equal to the shaft outside diameter Dₛ of the sensor adaptor 20, so that the sensor adaptor 20 can be inserted in that exit port 28. Typically, the exit ports 28 all have the same exit port diameter Dₚ, and the shaft outside diameter Dₛ is designed to be approximately equal to the exit port diameter Dₚ.

Also shown in FIG. 4, the sensor adaptor 20 extends into one of the exit ports 28 of the face mask 22 and thus couples the inner volume presented by the mask inner surface 68 to the gas sampling tube 24. Also in the embodiment of FIG. 4, the sensor adaptor 20 is formed separate from the face mask 22, and thus can be inserted, removed, and re-inserted into the face mask 22 by the practitioner, as many times as necessary. In this embodiment, the shaft 30 is inserted so that it frictionally engages the wall of the exit port 28 and thus remains in tight in the exit port 28 while the patient wears the face mask 22. The sensor adaptor 20 remains in the exit port 28, even while patient moves about, and thus provides a secure connection between the inner volume of the simple face mask 22, where the patient exhales carbon diode, and the gas sampling tube 24. If the practitioner needs to remove the sensor adaptor 20, he or she simply pulls the sensor adaptor 20 out of the exit port 28.

In another embodiment, the sensor adaptor 20 is formed integral with the face mask 22 and thus is permanently attached to the face mask 22 to provide an even more secure connection between the inner volume and the gas sampling tube 24. In yet another embodiment, the sensor adaptor 20 is formed integral with the gas sampling tube 24. However, forming the sensor adaptor 20 separate from the face mask 22 or gas sampling tube 24 provides an advantage because then the sensor adaptor 20 can be used with any existing face mask that includes exit ports, or any existing gas sampling tube. This saves a significant amount of resources, and money, as new face masks do not need to be purchased. The sensor adaptor 20 can also be used with various different devices capable of detecting the presence and/or absence the patient's end-tidal carbon dioxide. For example, the device can be an anesthesia machine, such as a machine referred to as D-fend™, or a device used in the rescue squad.

Another aspect of the invention provides a method of manufacturing an apparatus 66 including the sensor adaptor 20 which is coupled to the device which detects the patient's end-tidal carbon dioxide. The method includes providing the face mask 22 including the plurality of exit ports 28, and disposing the sensor adaptor 20 in one of the exit ports 28 of the face mask 22. The step of disposing the sensor adaptor 20 in the exit port 28 of the face mask 22 preferably includes inserting the adaptor tip 26 into the exit port 28 until the gripper 42 engages the mask outer surface 70. At this point, the adaptor tip 26 is disposed in the inner volume of the face mask 22. The sensor adaptor 20 is designed so that the adaptor length Iₐ is long enough to extend into the inner volume, but not so long that it touches the patient's face. When the sensor adaptor 20 is disposed in the exit port 28, the stopper end 52 prevents improper advancement of the shaft 30 into the inner volume of the face mask 22.

Another aspect of the invention provides a method for detecting the patient's end-tidal carbon dioxide. The method includes providing the face mask 22 and the sensor adaptor 20, as described above. The method next includes coupling the oxygen delivery tube 76 to the nasal opening 74 of the face mask 22, and disposing the face mask 22 over the nose and mouth of the patient to deliver oxygen to the patient. The method further includes coupling the connector end of the sensor adaptor 20 to the gas sampling tube 24 connected to the device monitoring the presence and/or absence of carbon dioxide in the inner volume of the simple face mask 22. The step of coupling the sensor adaptor 20 to the gas sampling tube 24 can be conducted either before or after the face mask 22 is disposed over the nose and mouth of the patient. When the sensor adaptor 20 is formed separate from the face mask 22, the step of coupling the sensor adaptor 20 to the face mask 22 includes inserting the adaptor tip 26 through one of the exit ports 28 of the face mask 22 until the stopper end 52 engages the mask outer surface 70. The sensor adaptor 20 remains securely connected to the face mask 22, and thus provides a convenient and reliable means to detect the presence or absence of end-tidal carbon dioxide in the inner volume of the face mask 22. The reliability provided by the sensor adaptor 20 allows for more rapid detection of airway obstruction, or other potential safety issues, and thus improves the quality of care provided to the patient.

Obviously, many modifications and variations of the present invention are possible in light of the above teachings and may be practiced otherwise than as specifically described while within the scope of the appended claims. In addition, although the term “diameter” is used to describe certain features of the invention, it is noted that those features do not necessary have a circular shape. Each of those features could comprise a rectangular shape, or another non-circular shape. Furthermore, although exemplary dimensions are disclosed for many components, it is noted that the components of the invention can comprise varies other dimensions.

What is claimed is:

1. A sensor adaptor for coupling a face mask to a gas sampling tube connected to a device detecting at least one of the presence and absence of end-tidal carbon dioxide, comprising:
a shaft extending from a first end to a second end, said shaft including a shaft outer surface presenting a shaft outside diameter being not greater than an exit port diameter of a face mask, said shaft including a shaft inner surface presenting a channel extending continuously from said first end to said second end for providing a continuous pathway from a face mask toward a gas sampling tube, said shaft including an adaptor tip adjacent said first end for being disposed in the exit port of the face mask,
a gripper surrounding said shaft adjacent said first end and presenting said adaptor tip between said gripper and said first end of said shaft, said gripper including a gripper outer surface presenting a gripper outside diameter being greater than said shaft outside diameter, and
a connector attached to said shaft for coupling said channel of said shaft to a gas sampling tube.

2. The sensor adaptor of claim 1 wherein said gripper includes a stopper end presenting a surface facing toward said first end for preventing advancement of the sensor adaptor into the exit port of the face mask.

3. The sensor adaptor of claim 1 wherein said gripper outer surface includes a plurality of grooves extending parallel to said shaft.

4. The sensor adaptor of claim 1 wherein said connector is attached to said second end of said shaft and includes a connector inner surface presenting a connector opening in fluid communication with said channel of said shaft, and said connector including at least one thread extending radially outwardly for engaging at least one thread of a gas sampling tube.

5. The sensor adaptor of claim 1 wherein said connector includes a female Luer lock.

6. The sensor adaptor of claim 1 wherein said shaft and said gripper and said connector are formed of a plastic material.

7. The sensor adaptor of claim 1 wherein said shaft and said gripper and said connector are formed integral with one another and comprise a single component.

8. A sensor adaptor for coupling a simple face mask to a gas sampling tube connected to a device detecting at least one of the presence and absence of end-tidal carbon dioxide, comprising:
a shaft extending from a first end to a second end, said shaft presenting a shaft length extending from said first end to said second end;
said shaft including a shaft outer surface presenting a cylindrical shape, said shaft outer surface presenting a shaft outside diameter being not greater than an exit port diameter of a simple face mask, said outside diameter being approximately 0.3 cm;
said shaft including a shaft inner surface facing opposite said shaft outer surface, said shaft inner surface presenting a channel having a cylindrical shape, said channel being unobstructed and extending continuously from said first end to said second end for providing a direct pathway for end-tidal carbon dioxide to travel toward a gas sampling tube;
said shaft including an adaptor tip adjacent said first end, said adaptor tip presenting said channel, said adaptor tip presenting an adaptor length being a portion of said shaft length, said adaptor length being 0.5 cm;
a gripper surrounding said shaft adjacent said first end, said gripper including a gripper inner surface presenting a gripper opening surrounding said shaft, said gripper including a gripper outer surface presenting a gripper outside diameter being greater than said shaft outside diameter, said gripper presenting a gripper length extending along a portion of said shaft length, said gripper length being 1.0 cm, said gripper outer surface presenting a plurality of grooves extending parallel to said shaft for allowing a practitioner to grip the sensor adaptor;
said gripper including a stopper end comprising a surface facing toward said first end of said shaft for preventing advancement of the sensor adaptor into the simple face mask, said stopper end and said first end of said shaft presenting said adaptor tip therebetween, said surface of said stopper end having a stopper diameter being greater than said shaft outside diameter (Dₛ);
a connector attached to said second end of said shaft, said connector including a connector inner surface presenting a connector opening in fluid communication with said channel of said shaft, said connector including a connector end being open for receiving an end of the gas sampling tube in said connector opening, said connector including at least one thread extending radially outwardly for engaging threads at the end of the gas sampling tube, said connector including a female Luer lock and the end of the gas sampling tube including a male Luer lock, said connector including a connector outer surface presenting a connector outside diameter being greater than said shaft outside diameter, said connector presenting a connector length being 2.4 cm, the distance between said connector and said gripper being 2.0 cm;
said connector including a pair of wings extending longitudinally along and radially outwardly;
said sensor adaptor presenting a total length extending from said open connector end to said first end of said shaft, said total length being 5.9 cm;
said shaft and said gripper and said connector being formed of a plastic material, and said plastic material being clear.

9. An apparatus for coupling to a device detecting at least one of the presence and absence of end-tidal carbon dioxide, comprising:
a face mask for disposing over a nose and mouth of a patient, said face mask including a mask inner surface presenting an inner volume, said face mask including a nasal opening for receiving an oxygen delivery tube, said face mask including a plurality of exit ports for allowing carbon dioxide to exit said inner volume, each of said exit ports presenting an exit port diameter;
a sensor adaptor for coupling said face mask to a gas sampling tube connected to the device detecting the end-tidal carbon dioxide;
the sensor adaptor including a shaft extending from a first end to a second end, said shaft including a shaft outer surface presenting a shaft outside diameter, said shaft including a shaft inner surface presenting a channel extending continuously from said first end to said second end for providing a continuous pathway from said face mask toward the gas sampling tube, said shaft including an adaptor tip adjacent said first end and being disposed in one of said exit ports of said face mask, said shaft outside diameter being not greater than the exit port diameter of said one exit port in which said shaft is disposed;
said sensor adaptor including a connector attached to said shaft; and
a gripper surrounding said shaft adjacent said first end and presenting said adaptor tip between said gripper and said first end of said shaft, said gripper including a gripper outer surface presenting a gripper outside diameter being greater than said shaft outside diameter, and said gripper including a stopper end preventing advancement of said sensor adaptor into said exit port;

10. The apparatus of claim 9 wherein said sensor adaptor and said face mask are formed integral with one another and comprise a single component.

11. The apparatus of claim 9 including a gas sampling tube coupled to said connector of said sensor adaptor.

12. The apparatus of claim 11 wherein an end of said gas sampling tube includes at least one thread, said connector is attached to said second end of said shaft and includes a connector inner surface presenting a connector opening in fluid communication with said channel of said shaft, and said connector includes at least one thread extending radially outwardly for engaging said at least one thread of said gas sampling tube.

13. The apparatus of claim 12 wherein said sensor adaptor and gas sampling tube are formed integral with one another and comprise a single component.

14. The apparatus of claim 9 wherein said face mask is a simple face mask.

15. An apparatus for coupling to a device detecting at least one of the presence and absence of end-tidal carbon dioxide, comprising:
a simple face mask for disposing over a nose and mouth of a patient, said simple face mask being formed of a plastic material, said plastic material being clear;
said simple face mask including a mask inner surface for facing a nose and mouth of the patient and a mask outer surface facing opposite said mask inner surface, said mask inner surface having a contour resembling the contour of a nose and mouth, and said mask inner surface presenting an inner volume between said mask inner surface and the nose and mouth of the patient;
said simple face mask including a nose portion with a nasal opening for receiving an oxygen delivery tube, the oxygen delivery tube presenting an airway being open for allowing oxygen to enter said inner volume;
said simple face mask including a pair of side portions disposed on opposite sides of said nose portion, each of said side portions including a plurality of exit ports for allowing the end-tidal carbon dioxide to exit the inner volume, each of said exit ports presenting an exit port diameter;
a sensor adaptor extending into one of said exit ports of said simple face mask for coupling said inner volume presented by said mask inner surface to a gas sampling tube connected to a device detecting at least one of the presence and absence of carbon dioxide in said inner volume;
said sensor adaptor including a shaft extending from a first end to a second end, said shaft presenting a shaft length extending from said first end to said second end;
said shaft including a shaft outer surface presenting a cylindrical shape, said shaft outer surface presenting a shaft outside diameter being approximately equal to said exit port diameter of said simple face mask, said shaft outside diameter being 0.3 cm;
said shaft including a shaft inner surface facing opposite said shaft outer surface, said shaft inner surface presenting a channel having a cylindrical shape, said channel being unobstructed and extending continuously from said first end to said second end for providing a direct pathway for end-tidal carbon dioxide to travel toward a standard gas sampling tube;
said shaft including an adaptor tip adjacent said first end and being disposed in said inner volume of said simple face mask, said adaptor tip presenting said channel, said adaptor tip presenting an adaptor length being a portion of said shaft length, said adaptor length being 0.5 cm;
said sensor adaptor including a gripper surrounding said shaft and being disposed adjacent said adaptor tip and outwardly of said mask outer surface, said gripper including a gripper inner surface presenting a gripper opening receiving said shaft, said gripper including a gripper outer surface presenting a gripper outside diameter being greater than said shaft outside diameter, said gripper presenting a gripper length being a portion of said shaft length, said gripper length being 1.0 cm, said gripper outer surface presenting a plurality of grooves extending parallel to said shaft;
said gripper including a stopper end presenting a surface facing toward said first end, said surface of said stopper end having a stopper diameter being greater than said shaft outside diameter and engaging said mask outer surface for preventing advancement of said sensor adaptor into said simple face mask, said stopper end and said first end presenting said adaptor tip therebetween;
said sensor adaptor including a connector attached to said second end of said shaft, said connector including a connector inner surface presenting a connector opening in fluid communication with said channel of said shaft, said connector including a connector end being open for receiving an end of the gas sampling tube in said connector opening, said connector including at least one thread extending radially outwardly for engaging threads of the gas sampling tube, said connector including a female Luer lock and the end of the gas sampling tube including a male Luer lock, said connector including a connector outer surface presenting a connector outside diameter being greater than said shaft outside diameter, said connector presenting a connector length being 2.4 cm, the distance between said connector end and said gripper being 2.0 cm;
said connector including a pair of wings extending longitudinally and radially outwardly;
said sensor adaptor presenting a total length extending from said open connector end to said first end of said shaft, said total length being 5.9 cm;
said shaft and said gripper and said connector of said sensor adaptor being formed of a plastic material, and said plastic material being clear.

16. A method for manufacturing an apparatus for coupling to a device detecting at least one of the presence and absence of end-tidal carbon dioxide, comprising:
providing a face mask including a mask outer surface and a mask inner surface presenting an inner volume, the face mask including a nasal opening for receiving an oxygen delivery tube, the face mask including a plurality of exit ports for allowing carbon dioxide to exit the inner volume, each of the exit ports presenting an exit port diameter;
disposing a sensor adaptor in one of the exit ports of the face mask, the sensor adaptor including a shaft extending from a first end to a second end, the shaft including a shaft outer surface presenting a shaft outside diameter being approximately equal to the exit port diameter of the face mask, the shaft including an inner surface presenting a channel extending continuously from the first end to the second end, and the shaft including an adaptor tip adjacent the first end, the sensor adaptor including a gripper surrounding the shaft adjacent the first end and presenting the adaptor tip between the gripper and the first end of the shaft, the gripper including a gripper outer surface presenting a gripper outside diameter being greater than the shaft outside diameter, and the sensor adaptor including a connector surrounding the shaft adjacent the second end; and
the step of disposing the sensor adaptor into the exit port of the face mask including inserting the adaptor tip into the exit port until the gripper engages the mask outer surface.

17. The method of claim 16 including the step of coupling the connector of the sensor adaptor to a gas sampling tube connected to the device detecting the end-tidal carbon dioxide.

18. The method of claim 16 wherein the gripper includes a gripper inner surface presenting a gripper opening, and including the step of forming the sensor adaptor by inserting the first end of the shaft into the gripper opening

19. A method for detecting at least one of the presence and absence of end-tidal carbon dioxide of a patient, comprising the steps of:
providing a face mask including a mask outer surface and a mask inner surface presenting an inner volume, the face mask including a nasal opening for receiving an oxygen delivery tube, the face mask including a plurality of exit ports for allowing carbon dioxide to exit the inner volume, each of the exit ports presenting an exit port diameter;
disposing a sensor adaptor in one of the exit ports of the face mask, the sensor adaptor including a shaft extending from a first end to a second end, the shaft including a shaft outer surface presenting a shaft outside diameter being approximately equal to the exit port diameter of the face mask, the shaft including an inner surface presenting a channel extending continuously from the first end to the second end, and the shaft including an adaptor tip adjacent the first end, the sensor adaptor including a gripper surrounding the shaft adjacent the first end and presenting the adaptor tip between the gripper and the first end of the shaft, the gripper including a gripper outer surface presenting a gripper outside diameter being greater than the shaft outside diameter, and the sensor adaptor including a connector surrounding the shaft adjacent the second end;
the step of disposing the sensor adaptor into the exit port of the face mask including inserting the adaptor tip into the exit port;
coupling an oxygen delivery tube to the nasal opening of the face mask;
disposing the face mask over the nose and mouth of the patient; and
coupling the connector end of the sensor adaptor to a gas sampling tube connected to a device for detecting at least one of the presence and absence of carbon dioxide present in the inner volume of the face mask.

20. The method of claim 19 wherein the step of disposing the sensor adaptor into the exit port of the face mask includes inserting the adaptor tip into the exit port until the gripper engages the mask outer surface;

21. A method for detecting at least one of the presence and absence of end-tidal carbon dioxide of a patient, comprising the steps of:
providing a simple face mask, the simple face mask including a mask inner surface for facing a nose and mouth of the patient and a mask outer surface facing opposite the mask inner surface, the mask inner surface having a contour resembling the contour of a nose and mouth, and the mask inner surface presenting an inner volume between the mask inner surface and the nose and mouth of the patient, the simple face mask including a nose portion with a nasal opening for receiving an oxygen delivery tube, the simple face mask including a pair of side portions disposed on opposite sides of the nose portion, the side portions including a plurality of exit ports for allowing the end-tidal carbon dioxide to exit the inner volume, and each of the exit ports presenting an exit port diameter;
providing a sensor adaptor, the sensor adaptor comprising:
a shaft extending from a first end to a second end, the shaft presenting a shaft length extending from the first end to the second end, the shaft including a shaft outer surface presenting a cylindrical shape, the shaft outer surface presenting a shaft outside diameter being not greater than the exit port diameter of the simple face mask, the outside diameter being 0.3 cm;
the shaft including a shaft inner surface facing opposite the shaft outer surface, the shaft inner surface presenting an channel having a cylindrical shape, the channel being unobstructed and extending continuously from the first end to the second end for providing a direct pathway for end-tidal carbon dioxide to travel toward a standard gas sampling tube;
the shaft including an adaptor tip adjacent the first end, the adaptor tip presenting the channel, the adaptor tip presenting an adaptor length being a portion of the shaft length, the adaptor length being 0.5 cm;
a gripper surrounding the shaft adjacent the first end, the gripper including a gripper inner surface presenting a gripper opening surrounding the shaft, the gripper including a gripper outer surface presenting a gripper outside diameter being greater than the shaft outside diameter, the gripper presenting a gripper length extending along a portion of the shaft length, the gripper length being 1.0 cm, the gripper outer surface presenting a plurality of grooves extending parallel to the shaft;
the gripper including a stopper end presenting a surface facing generally toward the first end;
a connector attached to the second end of the shaft, the connector including a connector inner surface presenting a connector opening in fluid communication with the channel of the shaft, receiving the shaft, the connector including a connector end being open for receiving an end of the gas sampling tube in the connector opening, the connector including at least one thread extending radially outwardly for engaging threads of the gas sampling tube, the connector including a female Luer lock and the end of the gas sampling tube including a male Luer lock, the connector including a connector outer surface presenting a connector outside diameter being greater than the shaft outside diameter, the connector presenting a connector length being 2.4 cm, the distance between the connector end and the gripper being 2.0 cm;
the connector end including a pair of wings extending longitudinally along and radially outwardly;
the sensor adaptor presenting a total length extending from the connector end to the first end of the shaft, the total length being 5.9 cm;
the shaft and the gripper and the connector end being formed of a plastic material, the plastic material being clear;
coupling an oxygen delivery tube to the nasal opening of the simple face mask;
coupling the sensor adaptor to the simple face mask by inserting the adaptor tip through one of the exit ports of the simple face mask until the stopper end engages the mask outer surface;
disposing the simple face mask over the nose and mouth of a patient; and
coupling the connector end of the sensor adaptor to a gas sampling tube connected to a device for detecting at least one of the presence and absence of carbon dioxide in the inner volume of the simple face mask.
